(19) United States
(12) Patent Application Publication
(10) Pub. No.: US 2012/0289834 A1
(43) Pub. Date: Nov. 15, 2012

(54) ULTRASOUND DIAGNOSTIC APPARATUS AND ULTRASOUND IMAGE PRODUCING METHOD

(76) Inventor: Tomoo SATO, Kanagawa (JP)

(21) Appl. No.: 13/469,808

(22) Filed: May 11, 2012

(30) Foreign Application Priority Data
May 12, 2011 (JP) 2011-107084

(51) Int. Cl. A61B 8/14

(52) U.S. Cl. 600/447

(57) ABSTRACT

An ultrasound diagnostic apparatus includes reception signal processors for amplifying reception signals outputted from a transducer array having received an ultrasonic echo from a subject and then A/D-converting the amplified reception signals to obtain reception data, the reception signal processors having an A/D conversion executable range where A/D conversion of the reception signals is possible, and a controller for controlling the reception signal processors to limit an A/D conversion execution range where A/D conversion is actually executed within the A/D conversion executable range according to amplitudes of the reception signals outputted from the transducer array.

### BACKGROUND OF THE INVENTION

The present invention relates to an ultrasound diagnostic apparatus and an ultrasound image producing method, and particularly to an ultrasound diagnostic apparatus producing an ultrasound image based on reception data acquired through amplification and A/D conversion of reception signals outputted from a transducer array having received ultrasonic echo reflected by a subject by reception signal processors.

In the medical field, ultrasound diagnostic apparatus employing ultrasound images have already been put to practical use. A typical ultrasound diagnostic apparatus for medical use transmits an ultrasonic beam from an array transducer of an ultrasound probe toward the inside of a subject, receives an ultrasonic echo from the subject on the array transducer, and electrically processes a reception signal corresponding to the received echo in an apparatus body so as to generate an ultrasound image.

As an example, JP 4-232888 A discloses an ultrasonic diagnostic system performing reception focusing, on which reception signals outputted from an array transducer having received ultrasonic echo components are each amplified by a preamplifier and subjected to A/D (analog/digital) conversion by an A/D converter to obtain digital reception data, and the obtained digital data are matched to one another in phase by imparting adequate delays to them, and as such added to one another so as to carry out the reception focusing.

According to the reception focusing process as disclosed, a sound ray signal is obtained as a well-focused ultrasonic echo, and the B-mode image signal which is tomographic image information on the inside of a subject is generated based on a plurality of sound ray signals obtained in a region under diagnosis.

### SUMMARY OF THE INVENTION

The A/D converter for A/D conversion of the reception signal is available in a wide variety of sorts. Among them, a successive-approximation type A/D converter is widely used for its compact circuit dimensions.

The successive-approximation type A/D converter compares a signal obtained by converting the value of a successive-approximation register into an analog signal and a reception signal outputted from each transducer and, based on the comparison result, successively changes the value of the successive-approximation register, repeating the comparison.

Specifically, as illustrated in FIG. 6, upon entry of a conversion start signal giving instructions to start the A/D conversion, the value of the successive-approximation register is changed at each clock pulse by bit from the most significant bit MSB to the least significant bit LSB of the A/D converter, and the signal obtained by converting the value of the successive-approximation register into an analog signal is successively compared with the reception signal outputted from each transducer. An n-bit A/D converter, for example, performs such comparison an n number of times until it completes A/D conversion of one reception signal, taking conversion time Tn that varies with the number of times the comparison is made.

Thus, even when the amplitude of the reception signal outputted from each transducer upon reception of ultrasonic echo is only a part of an A/D conversion executable range, the comparison is successively made over all the bits from the most significant bit MSB to the least significant bit LSB, requiring more conversion time and power consumption than necessary.

For production of an ultrasound image, the transducers of the transducer array output numerous reception signals as the ultrasonic beam scans a region under diagnosis, such that reduction of the conversion time and saving on power consumption for the A/D conversion of one reception signal result in significant reduction in processing time and power consumption required for the image processing performed by the ultrasound diagnostic apparatus.

An object of the present invention is to provide an ultrasound diagnostic apparatus and an ultrasound image producing method that resolve such problems of the past and enable faster image processing and reduced power consumption.

An ultrasound diagnostic apparatus according to the present invention comprises:

a transducer array;

a transmission driver for transmitting an ultrasonic beam from the transducer array toward a subject;

reception signal processors for amplifying reception signals outputted from the transducer array having received an ultrasonic echo from the subject and then A/D-converting the amplified reception signals to obtain reception data, the reception signal processors having an A/D conversion executable range where A/D conversion of the reception signals is possible;

an image producer for producing an ultrasound image based on the reception data obtained by the reception signal processors; and

a controller for controlling the reception signal processors to limit an A/D conversion execution range where A/D conversion is actually executed within the A/D conversion executable range according to amplitudes of the reception signals outputted from the transducer array.

An ultrasound image producing method according to the present invention comprises the steps of:

transmitting an ultrasonic beam from a transducer array toward a subject;

amplifying reception signals outputted from the transducer array having received an ultrasonic echo from the subject and then A/D-converting the amplified reception signals with reception signal processors having an A/D conversion executable range where A/D conversion of the reception signals is possible to obtain reception data;

producing an ultrasound image based on the obtained reception data; and

controlling the reception signal processors to limit an A/D conversion execution range where A/D conversion is actually executed within the A/D conversion executable range according to amplitudes of the reception signals outputted from the transducer array.

### BRIEF DESCRIPTION OF THE DRAWINGS

FIG. 1 is a block diagram illustrating a configuration of an ultrasound diagnostic apparatus according to an embodiment of the invention.

FIG. 2 is a block diagram illustrating an internal configuration of a reception signal processor used in the embodiment.

FIG. 3 is a block diagram illustrating an internal configuration of an A/D converter used in the embodiment.

FIG. 4 is a timing chart illustrating an operation of the A/D converter in the embodiment.

FIG. 5 is a graph illustrating a relation between echo signal and A/D conversion execution range.

FIG. 6 is a timing chart illustrating an operation of a conventional A/D converter.

### DETAILED DESCRIPTION OF THE INVENTION

In the following, an embodiment of the present invention is described in reference to the accompanying drawings.

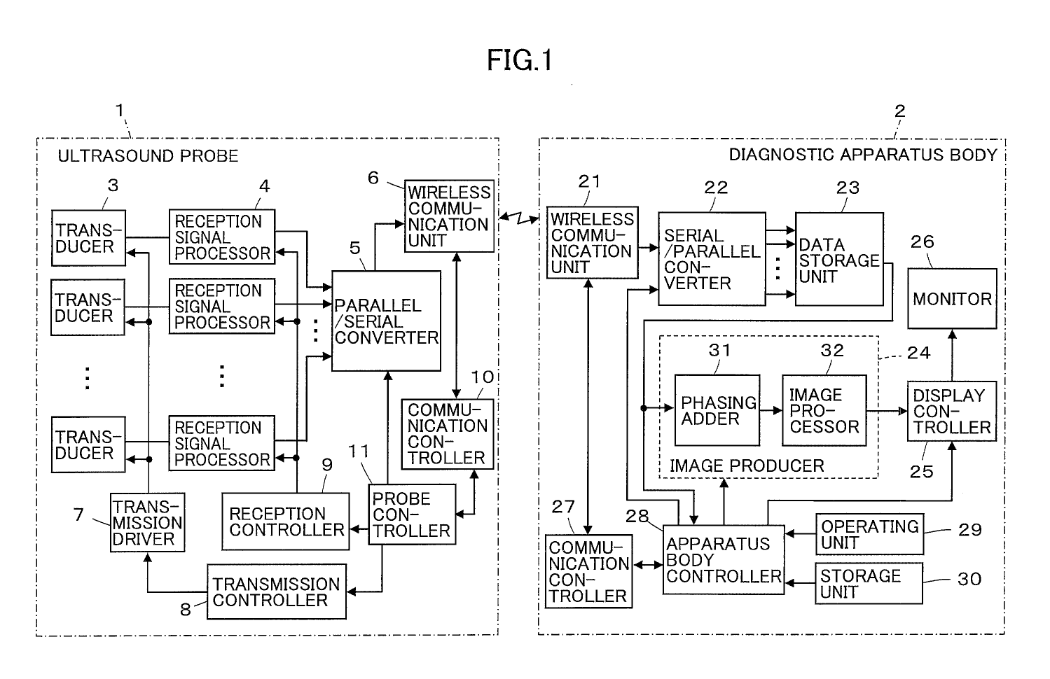

FIG. 1 shows the configuration of an ultrasound diagnostic apparatus according to the embodiment. The ultrasound diagnostic apparatus as shown includes an ultrasound probe 1 and a diagnostic apparatus body 2 connected with the ultrasound probe 1 through wireless communication.

The ultrasound probe 1 has a plurality of ultrasound transducers 3 unidimensionally or two-dimensionally arrayed, and connected to corresponding reception signal processors 4, respectively. The reception signal processors 4 are connected to a wireless communication unit 6 via a parallel/serial converter 5. The ultrasound transducers 3 are connected with a transmission controller 8 via a transmission driver 7, while the reception signal processors 4 are connected with a reception controller 9. The wireless communication unit 6 is connected with a communication controller 10. The parallel/serial converter 5, the transmission controller 8, the reception controller 9, and the communication controller 10 are connected with a probe controller 11.

Each of the ultrasound transducers 3 transmits ultrasound in accordance with a driving signal fed from the transmission driver 7, and receives an ultrasonic echo from a subject so as to output a reception signal. Each ultrasound transducer 3 is comprised of a vibrating element having a piezoelectic body and electrodes formed at both ends of the piezoelectric body, with examples of the material for the body including a piezoelectic ceramic typified by lead zirconate titanate (PZT), a polymeric piezoelectric material typified by polyvinylidene fluoride (PVDF), and a piezoelectric single crystal typified by lead magnesium niobate-lead titanate solid solution (PMN-PT).

If a pulsed voltage or a continuous wave voltage is applied across the electrodes of the vibrating element as above, the piezoelectic body expands and contracts, and ultrasound in pulse or continuous wave form is generated from the vibrating element. Ultrasounds generated from the individual vibrating elements are synthesized into an ultrasonic beam. In addition, each vibrating element expands and contracts during the reception of propagating ultrasound to generate an electric signal, and the electric signal is outputted as a reception signal representing the reception of ultrasound.

The transmission driver 7 includes, for instance, a plurality of pulse generators, and is adapted to modify, based on the transmission delay pattern as selected by the transmission controller 8, the delay amounts of the driving signals to be fed to the ultrasound transducers 3 so that ultrasounds transmitted from the ultrasound transducers 3 may form so wide an ultrasonic beam as to cover a specified tissue area in a subject, and then feed the driving signals to the transducers 3.

Each reception signal processor 4 generates a complex baseband signal by subjecting a reception signal outputted from the corresponding ultrasound transducer 3 to quadrature detection or quadrature sampling under the control of the reception controller 9, then performs sampling of the complex baseband signal to generate sample data including information on the tissue area, and feeds the sample data to the parallel/serial converter 5. The reception signal processors 4 may generate sample data by subjecting the data as acquired by the sampling of the complex baseband signal to data compression for low bit rate coding.

The parallel/serial converter 5 converts the sample data in parallel form as generated by the reception signal processors 4 into serial sample data.

The wireless communication unit 6 transmits the serial sample data by modulating a carrier based on the serial sample data to generate a transmission signal, and feeding the transmission signal to an antenna to thereby transmit radio waves from the antenna. Usable modulation methods include amplitude shift keying (ASK), phase shift keying (PSK), quadrature phase shift keying (QPSK), and sixteen quadrature amplitude modulation (16QAM).

The wireless communication unit 6 communicates with the diagnostic apparatus body 2 in a wireless manner so as not only to transmit sample data to the diagnostic apparatus body 2, but receive various control signals from the diagnostic apparatus body 2 to output the received control signals to the communication controller 10. The communication controller 10 controls the wireless communication unit 6 so that transmission of sample data may be performed at the radio field intensity for transmission as specified by the probe controller 11, and outputs the control signals as received by the wireless communication unit 6 to the probe controller 11.

The probe controller 11 controls individual components of the ultrasound probe 1 based on various control signals transmitted from the diagnostic apparatus body 2.

The ultrasound probe 1 has a battery included therein (not shown), from which power is fed to individual circuits in the ultrasound probe 1.

The ultrasound probe 1 may be an external probe, such as of a linear scanning type, of a convex scanning type, and of a sector scanning type, or a probe for endoscopic ultrasonography, such as of a radial scanning type.

The diagnostic apparatus body 2 has a wireless communication unit 21 connected to a data storage unit 23 via a serial/parallel converter 22, with the data storage unit 23 being connected to an image producer 24. The image producer 24 is connected to a monitor 26 via a display controller 25. The wireless communication unit 21 is also connected with a communication controller 27, and the serial/parallel converter 22, the data storage unit 23, the image producer 24, the display controller 25 and the communication controller 27 are connected with an apparatus body controller 28. The apparatus body controller 28 is in turn connected with an operating unit 29 used by an operator to perform input operations, and a storage unit 30 for storing operational programs.

The wireless communication unit 21 communicates with the ultrasound probe 1 in a wireless manner so as to transmit various control signals to the ultrasound probe 1. In addition, the wireless communication unit 21 outputs serial sample data by demodulating a signal received by an antenna.

The communication controller 27 controls the wireless communication unit 21 so that transmission of various control signals may be performed at the radio field intensity for transmission as specified by the apparatus body controller 28.

The serial/parallel converter 22 converts serial sample data outputted from the wireless communication unit 21 into parallel sample data. The data storage unit 23 is comprised of a memory, a hard disk or the like, and adapted to store the sample data as converted by the serial/parallel converter 22 for at least one frame.

The image producer 24 subjects the sample data as read frame by frame from the data storage unit 23 to reception focusing so as to produce an image signal representing an ultrasound diagnostic image. The image producer 24 includes a phasing adder 31 and an image processor 32.

The phasing adder 31 selects, in accordance with the reception direction as specified in the apparatus body controller 28, one reception delay pattern from among those stored in advance, and provides complex baseband signals represented by the sample data with their respective delays based on the selected reception delay pattern, then adds the delayed signals to thereby perform the reception focusing. The reception focusing allows a baseband signal (sound ray signal) as a well-focused ultrasonic echo.

The image processor 32 generates the B-mode image signal which is tomographic image information on a tissue in the subject based on a sound ray signal generated by the phasing adder 31. The image processor 32 includes a sensitivity time control (STC) device and a digital scan converter (DSC). The STC device corrects the sound ray signal for attenuation due to distance in accordance with the depth of the position where ultrasound was reflected. The DSC subjects the sound ray signal as corrected by the STC device to the conversion (raster conversion) into an image signal compatible with the conventional television signal scanning method, and to such image processing as grayscaling as required, so as to generate a B-mode image signal.

The display controller 25 controls the monitor 26 based on an image signal generated by the image producer 24 to display an ultrasound diagnostic image. The monitor 26 includes a display device such as an LCD, and is adapted to display an ultrasound diagnostic image under the control of the display controller 25.

The apparatus body controller 28 controls individual components of the diagnostic apparatus body 2 based on various instruction signals and the like inputted by an operator from the operating unit 29.

In the diagnostic apparatus body 2 as above, the serial/parallel converter 22, the image producer 24, the display controller 25, the communication controller 27, and the apparatus body controller 28 are implemented by a CPU associated with operational programs for giving the CPU instructions on various kinds of processing, while the above components may also be implemented by a digital circuitry. The operational programs are stored in the storage unit 30. As the storage unit 30, a recording medium such as a hard disk, a flexible disk, an MO, an MT, a RAM, a CD-ROM, a DVD-ROM, an SD card, a CF card, or a USB memory, a server, or the like may be used.

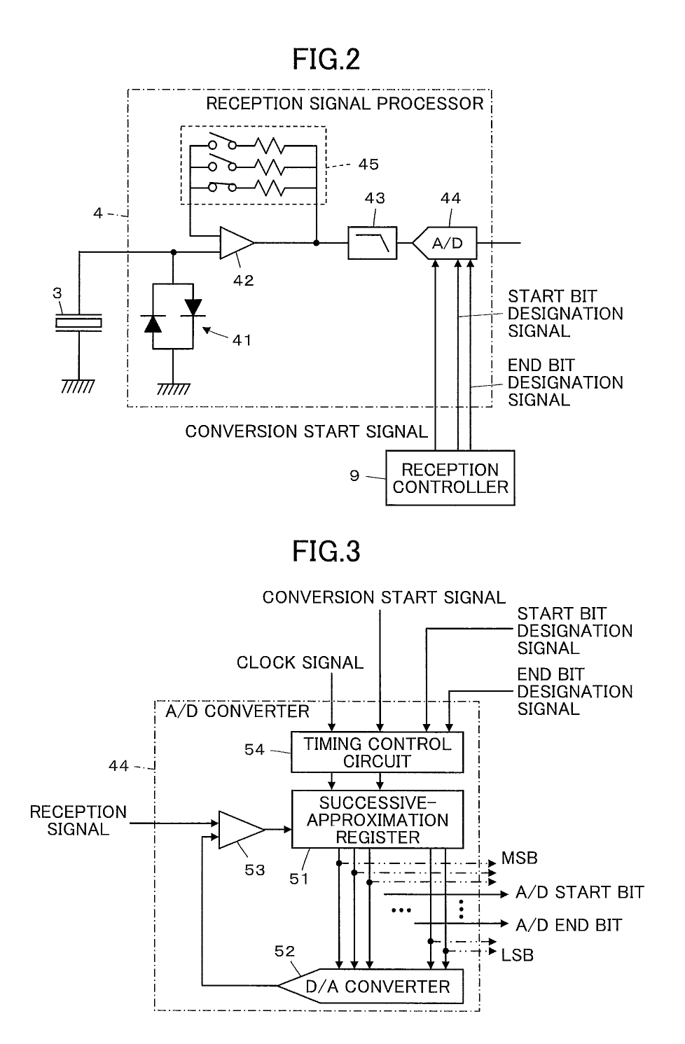

FIG. 2 shows the internal structure of each reception signal processor 4 in the ultrasound probe 1. Each reception signal processor 4 has a preamplifier 42 connected with the corresponding ultrasound transducer 3 via a clip circuit 41 for input protection, and an A/D converter 44 connected with an output terminal of the preamplifier 42 via a low pass filter 43. A gain setting circuit 45 is connected with the preamplifier 42 in a parallel manner.

The clip circuit 41 prevents a signal at a voltage exceeding a predetermined value from being inputted from the ultrasound transducer 3 to the preamplifier 42. The preamplifier 42 amplifies a reception signal outputted from the ultrasound transducer 3 with a gain set by the gain setting circuit 45 as appropriate to a subject and its site under diagnosis, and so forth. The low pass filter 43 removes the high frequency components which are not used for signal detection from the reception signal as amplified by the preamplifier 42. The A/D converter 44 converts the reception signal in analog form from which the high frequency components have been removed by the low pass filter 43 into a digital signal.

In the reception signal processor 4 with the structure as above, an A/D conversion executable range for A/D conversion of the reception signal by the reception signal processor 4 is determined, and A/D conversion of a reception signal is performed with a resolution depending on the dynamic range of the A/D converter 44.

Where the variation range of the amplitude of the reception signal outputted from the ultrasound transducer 3 is only a part of the A/D conversion executable range where the A/D conversion by the reception signal processor 4 is possible, even if the A/D conversion execution range actually used for execution of the A/D conversion is limited to a part thereof according to the amplitude of the reception signal by using only some of the bits among all the bits of the A/D converter 44 corresponding to the amplitude of the reception signal, it still enables accurate A/D conversion.

According to this embodiment, therefore, the reception controller 9 inputs a start bit designation signal for designating the A/D start bit and an end bit designation signal for designating the A/D end bit to the A/D converter 44 of the each reception signal processor 4 in addition to the conversion start signal that gives instructions to start the A/D conversion. The A/D converter 44 performs the A/D conversion of the reception signal by limiting the A/D conversion to the A/D conversion execution range determined by the A/D start bit and the A/D end bit designated respectively by the start bit designation signal and the end bit designation signal entered from the reception controller 9.

Each A/D converter 44 is a so-called successive-approximation type A/D converter, and comprises as illustrated in FIG. 3 a successive-approximation register 51, a D/A converter 52 for converting the value of the successive-approximation register 51 into an analog signal, and a comparator 53 for comparing the analog signal obtained through conversion by the D/A converter 52 and the reception signal entered via the low pass filter 43. The successive-approximation register 51 is connected to a timing control circuit 54 for changing the value of the successive-approximation register 51 based on the result of comparison made by the comparator 53.

In the A/D converter 44, the timing control circuit 54 successively changes the value of the successive-approximation register 51 as the comparator repeats comparison between the reception signal and the analog signal obtained by converting the value of the successive-approximation register 51. The value of the successive-approximation register 51 is not changed in all the bits thereof from the most significant bit MSB to the least significant bit LSB but only in the bits between the A/D start bit and the A/D end bit designated respectively by the start bit designation signal and the end bit designation signal entered from the reception controller 9 into the timing control circuit 54.

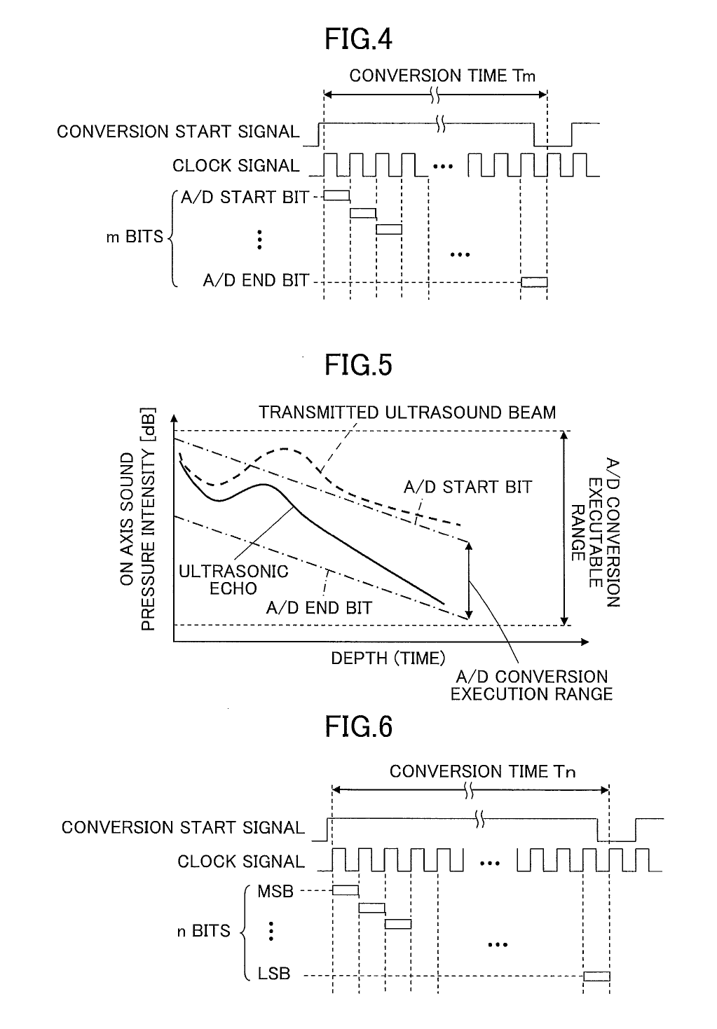

That is, as shown in FIG. 4, upon entry of the conversion start signal into the timing control circuit 54, the value of the successive-approximation register 51 is changed by bit every clock pulse from the A/D start bit designated by the start bit designation signal and the A/D end bit designated by the end bit designation signal, whereupon the signal obtained by converting the value of the successive-approximation register 51 into an analog signal and the reception signal are successively compared.

Thus, supposing that the A/D converter 44 is an n-bit A/D converter and that there are m bits (m<n) from the A/D start bit through the A/D end bit, completion of an A/D conversion would require time corresponding to n clock pulses when it is to be effected in the whole range containing the n bits from the most significant bit MSB to the least significant bit LSB whereas according to this embodiment, the A/D conversion of one reception signal is completed in m clock pulses. Thus, A/D conversion can be completed according to the embodiment in a shorter conversion time Tm than conversion time Tn that is required to effect the A/D conversion in the whole range containing the n bits from the most significant bit MSB to the least significant bit LSB and, accordingly, with reduced power consumption.

Next, the operation of this embodiment will be described.

First, prior to a diagnosis, an ultrasonic beam scans a subject in a pre-scan process. Specifically, the ultrasonic transducers 3 of the ultrasound probe 1 transmit an ultrasonic beam to the subject, and the reception signals outputted from the ultrasonic transducers 3 that have received an ultrasonic echo from the subject are supplied to the reception signal processors 4 to generate sample data, which are transmitted wirelessly to the diagnostic apparatus body 2 via the parallel/serial converter 13 and the wireless communication unit 14. The sample data received by the wireless communication unit 21 are converted into parallel data through the serial/parallel converter 22 and stored in the data storage unit 23. It is assumed that the A/D converter 44 of each reception signal processor 4 performs A/D conversion using all the bits from the most significant bit MSB to the least significant bit LSB without limitation in the A/D conversion execution range.

The apparatus body controller 28 recognizes the variation range of the amplitude of each reception signal outputted from the corresponding ultrasound transducer 3 based on the sample data stored in the data storage unit 23 and determines a sufficient A/D conversion execution range for effecting the A/D conversion of the reception signal based on the recognized variation range of the amplitude of the reception signal. The A/D conversion execution range is a range where the A/D conversion is actually effected by the A/D converter 44 of each reception signal processor 4. The A/D conversion execution range is set to a given bit width regardless of the measuring depth and may be defined by designating an A/D start bit and an A/D end bit.

Generally, in ultrasonic diagnosis, since the transmitted ultrasonic beam is attenuated as it travels through the subject, as the depth increases, the intensity of the ultrasonic beam reaching the depth decreases, as illustrated in FIG. 5. The ultrasonic echo reflected from points in the subject and returning to the ultrasound probe is also likewise attenuated as it travels. That is, as the measuring depth increases, the intensity of the ultrasonic echo generally decreases. As a result, the amplitude of the reception signal outputted from each ultrasound transducer 3 upon reception of the ultrasonic echo also varies with the measuring depth in substantially the same manner as the ultrasonic echo, decreasing as the measuring depth increases.

In order to receive the ultrasonic echo thus varying with the measuring depth, each of the reception signal processors 4 sets the A/D conversion executable range, where the A/D conversion by the reception signal processor 4 is possible, to a range corresponding to a range of intensity that covers all the intensities of the ultrasonic echoes returning from the points in a region to be measured. Further, the A/D conversion execution range is determined in accordance with the variation in intensity of the ultrasonic echo that changes with the measuring depth so as to cover a range of intensity having a width of intensity sufficiently encompassing the intensity of each ultrasonic echo that varies with each measuring depth and gradually decreasing as the measuring depth increases.

In FIG. 5, the A/D conversion executable range and the A/D conversion execution range are depicted on an ultrasonic echo intensity basis.

Thus, in order to specify the A/D conversion execution range that decreases as the measuring depth increases, the A/D start bit and the A/D end bit of the A/D conversion execution range are gradually reduced as the measuring depth increases.

The thus determined A/D start bit and A/D end bit of the A/D conversion execution range are wirelessly transmitted from the apparatus body controller 28 via the communication controller 27 and the wireless communication unit 21 to the ultrasound probe 1, received by the wireless communication unit 6, and then transmitted to the reception controller 9 via the communication controller 10 and the probe controller 11.

Upon the start of ultrasonography, the ultrasound transducers 3 transmit an ultrasonic beam in accordance with driving signals fed from the transmission driver 7 of the ultrasound probe 1, and the reception signals as outputted from the ultrasound transducers 3 having received ultrasonic echos from the subject are fed to the corresponding reception signal processors 4, respectively.

In each reception signal processor 4, the reception signal is amplified at the preamplifier 42, and inputted to the A/D converter 44 after high frequency components are removed from the signal at the low pass filter 43. The A/D converter 44 is supplied with the start bit designation signal and the end bit designation signal as well as the conversion start signal from the reception controller 9 and performs A/D conversion of the reception signal in the A/D conversion execution range determined by the A/D start bit and the A/D end bit designated respectively by the start bit designation signal and the end bit designation signal.

Specifically, the value of the successive-approximation register 51 is changed by bit every clock pulse from the A/D start bit to the A/D end bit by the timing control circuit 54, and the value of the successive-approximation register 51, now converted into an analog signal by the D/A converter 52, is successively compared with the corresponding reception signal thereby to achieve the A/D conversion.

Since the A/D conversion made in this process is performed within an A/D conversion execution range that is limited according to the amplitude of the reception signal instead of using all the bits from the most significant bit MSB to the least significant bit LSB of the A/D converter 44, the A/D conversion processing can be completed in a short time period.

Since the A/D start bit and the A/D end bit of the A/D conversion execution range designated by the reception controller 9 to the A/D converter 44 are gradually reduced as the measuring depth increases, an accurate A/D conversion of the reception signal is made possible by following the amplitude of the echo signal varying with the measuring depth.

Sample data is generated by such A/D conversion of a reception signal at the A/D converter 44 in each reception signal processor 4, and the sample data is made serial at the parallel/serial converter 5 before being transmitted from the wireless communication unit 6 to the diagnostic apparatus body 2 in a wireless manner. The sample data as received at the wireless communication unit 21 of the diagnostic apparatus body 2 is converted at the serial/parallel converter 22 into parallel data, then stored in the data storage unit 23. Further, the sample data are read from the data storage unit 23 frame by frame, and the image producer 24 generates image signals, based on which image signals the display controller 25 causes the monitor 26 to display an ultrasound diagnostic image.

As described above, since the A/D conversion is performed with the A/D conversion execution range limited according to the amplitude of the reception signals within the A/D conversion executable range where A/D conversion by the reception signal processors 4 is possible, reduction in conversion time and power consumption in A/D conversion of each reception signal can be achieved thereby enabling increased speed and reduced power consumption in the whole image production processing.

In the above embodiment, the A/D conversion execution range is specified by the reception controller 9 designating the A/D start bit and the A/D end bit to the A/D converter 44, but the A/D conversion execution range may otherwise be specified by designating the A/D start bit and a given bit width because the A/D conversion execution range is set to a given bit width irrespective of the measuring depth. Likewise, the A/D conversion execution range may be specified by designating the A/D end bit and a given bit width.

Although the apparatus body controller 28 of the diagnostic apparatus body 2 recognizes the variation in amplitude of each of the reception signals outputted from the ultrasound transducers 3 and determines the A/D conversion execution range for the A/D converter 44, the probe controller 11 of the ultrasound probe 1 may be, for example, adapted to effect the recognition of the variation in amplitude of each of the reception signals and the determination of the A/D conversion execution range of the A/D converter 44 based on the sample data produced by the reception signal processors 4.

In the above embodiment, the ultrasound probe 1 and the diagnostic apparatus body 2 are connected with each other through wireless communication, although the present invention is not limited thereto. It is also possible to connect the ultrasound probe 1 with the diagnostic apparatus body 2 via a connection cable. In that case, the wireless communication unit 6 and the communication controller 10 of the ultrasound probe 1, the wireless communication unit 21 and the communication controller 27 of the diagnostic apparatus body 2, and so forth are unnecessary.

What is claimed is:

1. An ultrasound diagnostic apparatus comprising:
a transducer array;
a transmission driver for transmitting an ultrasonic beam from the transducer array toward a subject;
reception signal processors for amplifying reception signals outputted from the transducer array having received an ultrasonic echo from the subject and then A/D-converting the amplified reception signals to obtain reception data, the reception signal processors having an A/D conversion executable range where A/D conversion of the reception signals is possible;
an image producer for producing an ultrasound image based on the reception data obtained by the reception signal processors; and
a controller for controlling the reception signal processors to limit an A/D conversion execution range where A/D conversion is actually executed within the A/D conversion executable range according to amplitudes of the reception signals outputted from the transducer array.

2. The ultrasound diagnostic apparatus according to claim 1,
wherein the reception signal processors comprise successive-approximation type A/D converters for A/D-converting the reception signals,
the controller controlling the A/D converters to limit the A/D conversion execution range.

3. The ultrasound diagnostic apparatus according to claim 2, wherein the controller designates to the A/D converters a start bit of the A/D conversion execution range determined according to the amplitudes of the reception signals outputted from the transducer array.

4. The ultrasound diagnostic apparatus according to claim 3, wherein the controller gradually reduces the start bit of the A/D conversion execution range as a measuring depth increases.

5. The ultrasound diagnostic apparatus according to claim 2, wherein the controller sets the A/D conversion execution range to a given bit width irrespective of a measuring depth.

6. The ultrasound diagnostic apparatus according to claim 4, wherein the controller sets the A/D conversion execution range to a given bit width irrespective of the measuring depth.

7. The ultrasound diagnostic apparatus according to claim 2, wherein the controller determines the A/D conversion execution range according to the amplitudes of the reception signals obtained by pre-scan.

8. The ultrasound diagnostic apparatus according to claim 4, wherein the controller determines the A/D conversion execution range according to the amplitudes of the reception signals obtained by pre-scan.

9. The ultrasound diagnostic apparatus according to claim 5, wherein the controller determines the A/D conversion execution range according to the amplitudes of the reception signals obtained by pre-scan.

10. The ultrasound diagnostic apparatus according to claim 6, wherein the controller determines the A/D conversion execution range according to the amplitudes of the reception signals obtained by pre-scan.

11. The ultrasound diagnostic apparatus according to claim 7, wherein the controller determines the A/D conversion execution range according to the amplitudes of the reception signals obtained by pre-scan.

12. The ultrasound diagnostic apparatus according to claim 2,
wherein the A/D converters each include a successive-approximation register, a D/A converter for converting a value of the successive-approximation register into an analog signal, a comparator for comparing the analog signal obtained through conversion by the D/A converter and a corresponding one of the reception signals outputted from the transducer array, and a timing control circuit for changing the value of the successive-approximation register based on a comparison result of the comparator,
the controller controlling the timing control circuit to limit the A/D conversion execution range.

13. The ultrasound diagnostic apparatus according to claim 7,
wherein the A/D converters each include a successive-approximation register, a D/A converter for converting a value of the successive-approximation register into an analog signal, a comparator for comparing the analog signal obtained through conversion by the D/A converter and a corresponding one of the reception signals outputted from the transducer array, and a timing control circuit for changing the value of the successive-approximation register based on a comparison result of the comparator,
the controller controlling the timing control circuit to limit the A/D conversion execution range.

14. The ultrasound diagnostic apparatus according to claim 13,
wherein the A/D converters each include a successive-approximation register, a D/A converter for converting a value of the successive-approximation register into an analog signal, a comparator for comparing the analog signal obtained through conversion by the D/A converter and a corresponding one of the reception signals outputted from the transducer array, and a timing control circuit for changing the value of the successive-approximation register based on a comparison result of the comparator,
the controller controlling the timing control circuit to limit the A/D conversion execution range.

15. The ultrasound diagnostic apparatus according to claim 2, wherein the reception signal processors each comprise a preamplifier for amplifying a corresponding one of the reception signals outputted from the transducer array and a low pass filter for removing a high-frequency component not used for signal detection from the corresponding one of the reception signals amplified by the preamplifier,
each of the A/D converters converting the corresponding one of the reception signals from which the high-frequency component has been removed by the corresponding low pass filter into a digital signal.

16. The ultrasound diagnostic apparatus according to claim 7,
wherein the reception signal processors each comprises a preamplifier for amplifying a corresponding one of the reception signals outputted from the transducer array and a low pass filter for removing a high-frequency component not used for signal detection from the corresponding one of the reception signals amplified by the preamplifier,
each of the A/D converters converting the corresponding one of the reception signals from which the high-frequency component has been removed by the corresponding low pass filter into a digital signal.

17. The ultrasound diagnostic apparatus according to claim 13,
wherein the reception signal processors each comprises a preamplifier for amplifying a corresponding one of the reception signals outputted from the transducer array and a low pass filter for removing a high-frequency component not used for signal detection from the corresponding one of the reception signals amplified by the preamplifier,
each of the A/D converters converting the corresponding one of the reception signals from which the high-frequency component has been removed by the corresponding low pass filter into a digital signal.

18. The ultrasound diagnostic apparatus according to claim 16,
wherein the reception signal processors each comprises a preamplifier for amplifying a corresponding one of the reception signals outputted from the transducer array and a low pass filter for removing a high-frequency component not used for signal detection from the corresponding one of the reception signals amplified by the preamplifier,
each of the A/D converters converting the corresponding one of the reception signals from which the high-frequency component has been removed by the corresponding low pass filter into a digital signal.

19. An ultrasound image producing method comprising the steps of:
transmitting an ultrasonic beam from a transducer array toward a subject;
amplifying reception signals outputted from the transducer array having received an ultrasonic echo from the subject and then A/D-converting the amplified reception signals with reception signal processors having an A/D conversion executable range where A/D conversion of the reception signals is possible to obtain reception data;
producing an ultrasound image based on the obtained reception data; and
controlling the reception signal processors to limit an A/D conversion execution range where A/D conversion is actually executed within the A/D conversion executable range according to amplitudes of the reception signals outputted from the transducer array.
